(19) United States
(12) Patent Application Publication
(10) Pub. No.: US 2013/0197366 A1
(43) Pub. Date: Aug. 1, 2013

(54) SYSTEMS AND METHODS FOR INTRAVASCULAR ULTRASOUND IMAGING

(71) Applicants: Kenneth Wayne Rigby, Clifton Park, NY (US); David Martin Mills, Niskayuna, NY (US); Ying Fan, Niskayuna, NY (US)

(72) Inventors: Kenneth Wayne Rigby, Clifton Park, NY (US); David Martin Mills, Niskayuna, NY (US); Ying Fan, Niskayuna, NY (US)

(73) Assignee: GENERAL ELECTRIC COMPANY, SCHENECTADY, NY (US)

(21) Appl. No.: 13/362,394

(22) Filed: Jan. 31, 2012

(51) Int. Cl. A61B 8/12

(52) U.S. Cl. 600/447; 600/443

(57) ABSTRACT

Embodiments of catheters, imaging systems and methods for intravascular ultrasound imaging are presented. At least one miniaturized transducer element adapted to be inserted into a vascular structure and configured to produce signals for use in generating one or more ultrasound images of a desired region within the vascular structure is used. Further, one or more apodizing structures operably coupled to the transducer element and configured to decrease the responsiveness of the transducer element at one or more boundaries of the transducer element with respect to the center of the transducer element are used. Particularly, the apodizing structures reduce sidelobe amplitude of an ultrasound beam profile in the region of interest, thereby generating improved ultrasound images.

### BACKGROUND

Embodiments of the present technique relate generally to diagnostic imaging, and more particularly to systems and methods for reducing artifacts in intravascular ultrasound images for improved image quality.

Ultrasonic imaging is a well-known imaging modality that employs ultrasonic acoustic energy generated by ultrasonic transducers. Particularly, ultrasound imaging is commonly used in industrial imaging for non-destructive evaluation and allows visualizing through various materials, components, or systems to detect corresponding features and characteristics. Ultrasound imaging has also found widespread use in many medical applications.

Several conventional industrial ultrasound systems employ large single-element transducers, for example, having a size of about a few centimeters. Further, conventional ultrasound systems used for medical imaging often employ an array of transducer elements. These ultrasound systems process data received from the multiple transducer elements to generate diagnostic images. In many medical imaging applications, the typical dimension of the array of transducer elements is also about a few centimeters. In other applications, the ultrasound systems may employ as few as a single transducer element that may be mechanically rotated or moved to allow for a greater imaged region. In these applications, such as intravascular or transesophageal imaging, the typical size of the transducer element is about a few millimeters, or even less.

Medical diagnostic ultrasound, for example, is an imaging modality that employs ultrasonic transducers that generate ultrasound pulses to probe acoustic properties of biological tissues and produce corresponding images. More specifically, diagnostic ultrasound systems visualize muscles, tendons, and other internal organs to assess their size, structure and any pathological lesions using real-time or near real-time images. Further, diagnostic ultrasound also finds use in therapeutics where an ultrasound probe is used to guide interventional procedures such as biopsies.

In order to improve the usefulness of resulting images, some of these ultrasound systems employ apodizing functions as described herein. In particular, certain ultrasound systems employ aperture apodization to reduce the response of an associated aperture at its boundaries. “Apodization,” also known as “shading,” refers to smoothly reducing the response of an aperture at its boundaries. For systems using a single transducer element, the aperture is the active face of the transducer element. For systems using an array of transducer elements, the aperture is the active face of the array of transducer elements. Apodization is typically used to modify a spatial response, the point-spread-function or “beam pattern” of an aperture, in an imaging system. The beam pattern is conventionally divided into mainlobe and sidelobe regions. When an aperture is apodized with a suitable apodization function, the mainlobe widens and the amplitude of the response in the sidelobe region is reduced.

Low sidelobe amplitude is desirable because it improves the usefulness of the reconstructed image in many commonly encountered imaging situations. For example, low sidelobe amplitudes are desirable for mitigating artifacts that arise when relatively strong reflectors are present in the sidelobe region for a particular beam direction and relatively weak reflectors are present in the mainlobe region. In many applications, the benefit arising from reducing the sidelobe amplitude outweighs the undesirable widening of the mainlobe.

Accordingly, certain ultrasound systems using arrays of transducer elements are known to employ electronic apodization by electronically attenuating signals transmitted and received from individual elements of multi-element transducer arrays to reduce sidelobe artifacts. For large single-element transducers intended for industrial imaging, a number of structural apodization techniques have been proposed, such as the use of an acoustic lens, a pattern of grooves, varying the amount and polarization of piezoelectric material, and by using a resistive or patterned electrode.

Intravascular Ultrasound (IVUS) imaging, however, employs miniaturized transducer elements. IVUS systems use these miniaturized transducer elements while diagnosing blocked blood vessels to provide information that aid medical practitioners in procedures such as angiography and stent placement to restore or increase blood flow to a desired region. Further, IVUS imaging systems have also been used to determine the existence as well as the nature and extent of intravascular obstructions, stenosis, or atheromatous plaque build-up at particular locations within the blood vessels. Particularly, IVUS imaging systems are used to visualize segments of a vascular system that may be difficult to visualize using other imaging techniques, such as angiography, due to cardiac movement or obstructions.

To that end, in certain implementations such as during IVUS imaging, a catheter including a miniaturized ultrasound probe is inserted into, or proximal, the region of interest (ROI) in a confined space such as a coronary vessel where high-frequency sound waves reflect off tissue or vessel walls. The reflected sound waves are used to create a cross-sectional image from within the vessel that aids in visualizing the corresponding structures. The transducers used in the IVUS imaging, however, are much smaller than the transducers used in industrial and other types of medical imaging. Specifically, in certain implementations, the IVUS transducers are about 0.4-1 millimeter in size.

While conventional IVUS systems having small transducers have been used in visualizing extremely small structures, the accuracy of the visualization affects the medical diagnosis and treatment prescribed to the patient. Conventional IVUS systems using miniaturized single-element transducers have not employed the structural apodization techniques described herein. As IVUS imaging is particularly vulnerable to sidelobe artifacts because of the approximately circular geometry of the tissue structures of interest in intravascular imaging, there are continuing needs for improved quality IVUS imaging.

### BRIEF DESCRIPTION

Certain aspects of the present technique present an intravascular ultrasound catheter including at least one miniaturized transducer element adapted for use in an intravascular region of interest of a subject. Particularly, the miniaturized transducer element is configured to produce signals for use in generating one or more ultrasound images of the intravascular region of interest of the subject. The catheter further includes one or more apodizing structures operably connected to the transducer element. The apodizing structures are configured to decrease the responsiveness of the transducer element at one or more boundaries of the transducer element with respect to the center of the transducer element for reducing sidelobe amplitude of an ultrasound beam profile in the region of interest, thereby generating improved ultrasound images.

Certain further aspects of the present technique include an intravascular ultrasound imaging system including a catheter. The catheter further includes at least one miniaturized transducer element adapted to be inserted into a vascular structure, and configured to produce signals for use in generating one or more ultrasound images of a desired region within the vascular structure. The system further includes one or more apodizing structures operably coupled to the transducer element. The apodizing structures are configured to decrease the responsiveness of the transducer element at one or more boundaries of the transducer element with respect to the center of the transducer element for reducing a sidelobe amplitude of an ultrasound beam profile used for imaging the desired region. The system also includes a processing unit operably coupled to the catheter. The processing unit processes the signals generated by the transducer element for generating the one or more ultrasound images of the desired region within the vascular structure such that the one or more ultrasound images have reduced artifacts.

Certain other aspects of the present technique are drawn to an ultrasound imaging method. One or more signals are generated for imaging a desired region of a vascular structure using an intravascular transducer. The transducer includes at least one miniaturized transducer element and one or more apodizing structures configured to decrease the responsiveness of the transducer element at one or more boundaries of the transducer element with respect to the center of the transducer element for reducing a sidelobe amplitude of an ultrasound beam profile used for imaging the desired region. Further, one or more improved ultrasound images of the desired region are reconstructed using data acquired using the apodized transducer element.

### DRAWINGS

These and other features, and aspects of embodiments of the present technique will become better understood when the following detailed description is read with reference to the accompanying drawings in which like characters represent like parts throughout the drawings, wherein:

FIG. 1 is a schematic representation of an exemplary IVUS imaging system, in accordance with certain aspects of the present system;

FIG. 2 is a schematic representation of an exemplary transducer for use in improved IVUS imaging, in accordance with certain aspects of the present technique;

FIG. 3 is a schematic representation of another exemplary transducer for use in improved IVUS imaging, in accordance with certain aspects of the present technique;

FIG. 4 is a schematic representation of another exemplary transducer for use in improved IVUS imaging, in accordance with certain aspects of the present technique;

FIG. 5 is a flow diagram illustrating an exemplary method for improved intravascular ultrasound imaging using apodizing structures, in accordance with certain aspects of the present technique;

FIG. 6 is an illustration of the geometry of an exemplary model of a blood vessel and a rotating single-element transducer used for IVUS imaging, in accordance with certain aspects of the present technique;

FIG. 7 is an illustration of the geometry of another exemplary model depicting eight point-like scatterers for use in describing the appearance of the image artifacts in the blood vessel model of FIG. 6, in accordance with certain aspects of the present technique;

FIGS. 8-9 are illustrations of simulated images produced by the models illustrated in FIG. 6 and FIG. 7, respectively, when the single-element transducer is not apodized;

FIGS. 10-11 are illustrations of simulated images produced by the models illustrated in FIG. 6 and FIG. 7, respectively, when the single-element transducer is apodized;

FIGS. 12-13 are illustrations of simulated images produced by the models illustrated in FIG. 6 and FIG. 7, respectively, using an unapodized single-element transducer when the transducer rotation axis is not aligned with the center of the simulated blood vessel; and

FIGS. 14-15 are illustrations of simulated images produced by the models illustrated in FIG. 6 and FIG. 7, respectively, using an apodized single-element transducer when the transducer rotation axis is not aligned with the center of the simulated blood vessel.

### DETAILED DESCRIPTION

The following description presents intravascular ultrasound (IVUS) systems for reducing artifacts in ultrasound images using apodization functions for improved image quality, and methods of making and using such improved IVUS systems. The inventors of the present system realized the need for improved imaging quality during intravascular imaging. For example, it was realized that a metal strut of a coronary artery stent or calcium deposits formed in some diseased arteries may prove to be more reflective than most tissue structures and typically contributes to image artifacts when such strong reflectors are present in the sidelobe region of the ultrasound beam pattern for a particular imaging direction. Similarly, while imaging blood vessels, the media layer weakly reflects ultrasound energy as compared to the surrounding intima and adventia layers. In such scenarios, the net response of an unapodized aperture may be dominated by the reflectors in the sidelobe region. The brightness of the displayed pixel, thus, may not be representative of the true strength of the reflected ultrasound at an image source point, in turn leading to artifacts in the resulting ultrasound images.

Conventional ultrasound systems employ apodization functions with large transducer elements or large arrays of transducer elements. However, such apodization was not previously employed with the much smaller transducers used in the IVUS applications. IVUS imaging systems are not known to use electronic or any other apodization functions, presumably owing to difficulties in implementing apodization in a high-frequency, extremely small-sized IVUS transducer element in desired geometries. Furthermore, during minimally invasive IVUS imaging, the small-sized IVUS transducer elements need to operate in confined biological environments where tissue fluids, blood or digested food may not only obstruct the path of visible light but can also erode or electrically short the imaging devices.

Accordingly, embodiments of the present system and techniques are directed to improved imaging applications that heretofore may not have not been desired, or deemed necessary. Particularly, certain embodiments illustrated herein describe systems and the methods that efficiently mitigate artifacts in the ultrasound images caused by significant sidelobe amplitudes in beam patterns used for IVUS imaging in confined environments such as in human vascular structures. To that end, the embodiments presented herein describe one or more apodizing structures adapted to reduce side-lobe amplitude levels, which in turn reduces artifacts in the corresponding reconstructed images for improved image quality.

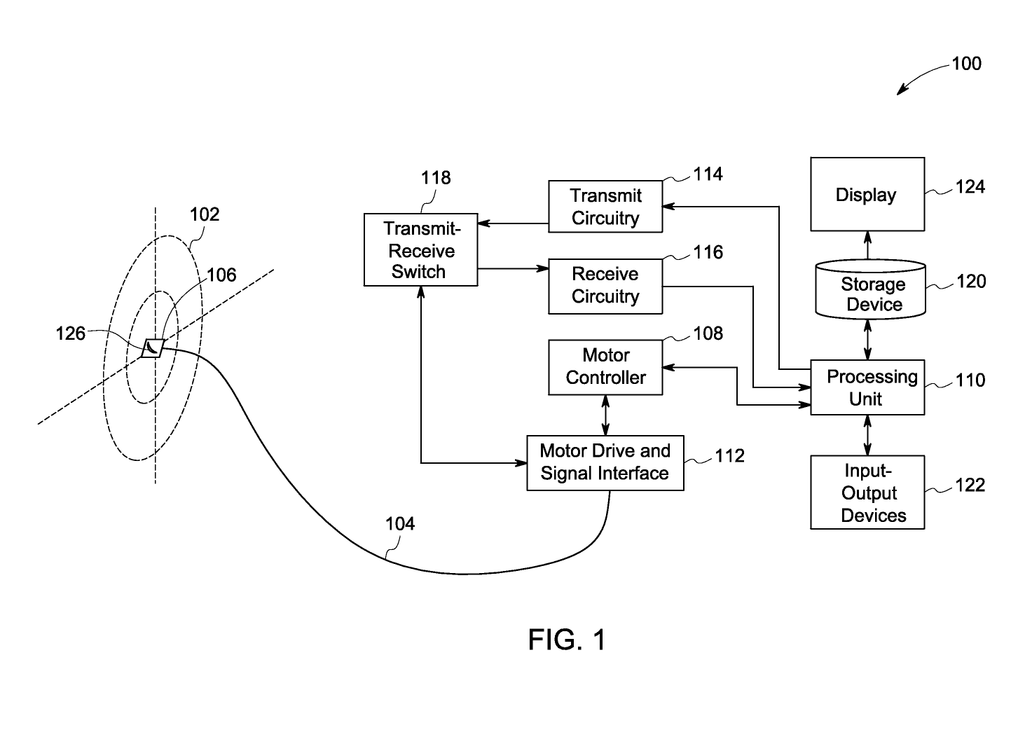

One embodiment of the present system, thus, incorporates appropriate electronic control circuitry or other apodization functions typically applied to large transducers, to the small-sized transducer elements directed to IVUS applications. An exemplary environment that is suitable for practicing various implementations of the present system is described in the following sections with reference to FIG. 1.

FIG. 1 illustrates an exemplary ultrasound system 100, for example, for use in intravascular imaging. To that end, in one embodiment, the system 100 includes a catheter 104 including a miniaturized single element transducer 106 adapted for use in a confined medical or surgical environment such as a body cavity, orifice or a blood vessel 102. The dimension of the miniaturized transducer 106, in one example, is about 0.5 millimeter. The size of the transducer 106, in other examples, is in the range of 0.25 to 1.0 millimeter. Further, the transducer 106 may be flat or curved, disc-, block-, spherical- or ring-shaped based on specific imaging requirements. In certain embodiments, the shape and size of the transducer 106 is selected such that the transducer 106 is suitable for being inserted or placed inside a patient's body without significant tissue disruption.

The embodiment illustrated in FIG. 1 presents a single-element transducer 106 configured to rotate mechanically about the catheter 104, offering side-looking capabilities. However, in certain other embodiments, the catheter 104 includes a multi-element array of transducers such that the resultant imaging can provide a radial cross-sectional image of the vessel wall. The single-element transducer or multi-element array of transducers may also be configured to produce a forward-looking image.

Accordingly, in certain embodiments, the catheter 104, including the miniaturized transducer 106, is placed inside the blood vessel 102 to be imaged. For example, in one embodiment, the catheter is inserted into the blood vessel 102 through one or more small incisions to reduce recovery time. In one example, the transducer 106 is rotated inside the blood vessel 102 under control of a motor controller 108 and/or a processing unit 110 through a motor drive and signal interface 112. Particularly, the motor controller 108 and/or the processing unit 110 provide control and timing signals for controlling the delivery sequence of the different ultrasound pulses, frequency of delivering the pulses, a time delay between two different pulses, beam intensity, and/or other imaging system parameters.

In certain embodiments, the system 100 further includes transmit circuitry 114 and receive circuitry 116, which are electrically connected to the rotatable transducer 106 through a transmit-receive switch 118 and the motor drive and signal interface 108. In one embodiment, the transmit circuitry 114, under control of processing unit 110, generates a pulsed electrical signal to drive the transducer 106 to emit ultrasound energy into a desired ROI (not shown). At least a portion of the emitted ultrasound energy is reflected from one or more scatterers in the ROI to produce reflected ultrasound energy that returns to the transducer 106, which in turn converts the reflected ultrasound energy into electrical signals. These electrical signals pass through the motor drive and signal interface 112 and the transmit-receive switch 118 to the receive circuitry 116 for further processing.

Particularly, in one embodiment, the received electrical signals are provided to the processing unit 110 that processes the received signals according to a plurality of selectable ultrasound modalities in near real-time and/or off-line mode. To that end, the processing unit 110 includes devices such as one or more application-specific processors, digital signal processors, microcomputers, microcontrollers, Application Specific Integrated Circuits (ASICs), Field Programmable Gate Arrays (FPGAs), or other suitable devices in communication with other components of the system 100.

In certain embodiments, the processing unit 110 stores the received signals along with the delivery sequence, frequency, time delay, beam intensity and/or imaging system parameters and other operational data in a storage device 120 for further processing. To that end, the storage device 120 includes devices such as a random access memory, a read-only memory, a disc drive, solid-state memory device, and/or a flash memory. Additionally, the processing unit 110 may communicate the determined values to the motor controller 108 operatively coupled to a motor (not shown) by the motor drive and signal interface 112.

In certain embodiments, the motor controller 108 uses the values received from the processing unit 110 to configure the transducer 106 to rotate about the longitudinal axis of the catheter 104, for example, via a rotatable driveshaft. Particularly, the motor controller 108 controls a rate of rotation of the transducer 106 based on specific imaging requirements. To that end, in certain embodiments, the motors controller 118 configures the motor drive and signal interface 112 to rotate the transducer 106 while allowing electrical signals to pass between the rotating transducer 106 and the stationary components of the system 100. Although FIG. 1 illustrates the motor controller 108 as an independent entity, in certain embodiments, the motor controller 108 is implemented as part of the processing unit 110.

Further, in certain embodiments, the processing unit 110 is coupled to one or more user input-output devices 122, such as a keyboard, touchscreen, microphone, mouse, buttons, and/or switches for receiving commands and inputs from an operator. In one example, the processing unit 110 allows the operator to select one or more imaging regions of interest, and/or imaging parameters, for example, using a graphical user interface on a local or remote display device 124 communicatively coupled to the processing unit 110 and/or the input output devices 122. The imaging parameters, for example, include a rate of rotation of the transducer 106, a velocity or length of the pullback of the catheter 104, and one or more desired properties of the images.

The processing unit 110, in one embodiment, conveys operator inputs to one or more of the transmit circuitry 114, and the motor controller 108. Accordingly, the transducer 106 rotating under the control of the motor controller 108 emits ultrasound pulses towards one or more desired portions of the region surrounding the transducer 106 to allow generation of a plurality of imaging lines. These imaging lines may be used collectively to reconstruct a radial cross-sectional image of the desired ROI, such as the walls of the blood vessel 102 and the tissue surrounding the blood vessel 102.

In one example, the processing unit 110 displays corresponding patient data including ultrasound images for review, diagnosis, analysis, and treatment. In another example, the processing unit 110 stores the ultrasound images for later review and analysis or communicates the images to another location for further review. Further, in one embodiment, a user employs the generated images, for example, for detecting a disease condition such as presence of plaque or other blockages, and/or to aid in deploying a vascular stent.

As detailed herein, the IVUS images are subject to artifacts that diminish the image quality. The brightness of the displayed pixel may not be representative of the true strength of the ultrasound reflected from the desired ROI in certain scenarios, thus leading to erroneous reconstruction of clinically relevant parameters in the resulting ultrasound images. Particularly, inaccurate estimations of the clinically relevant parameters, such as features that indicate a nature and extent of blockage in a particular ROI, using erroneous images may lead to incorrect diagnosis, which in turn may adversely affect patient treatment and health. Accordingly, the present system generates high quality IVUS images that are substantially free from artifacts to allow a clinician to trust the values computed or estimated from the reconstructed images.

A single-element transducer used in a conventional IVUS system is devoid of apodization. Conventional IVUS transducer elements include uniformly thick backing, electrode and matching layers configured to emit a nearly equal amplitude sound wave across the face of the transducer element when operated in transmit mode, and to be nearly equally sensitive across the face of the transducer element to incident sound when operated in receive mode. Use of layers of uniform thickness simplifies the construction of the transducer, reduces costs, maximizes the transducer element's electrical sensitivity, and/or maximizes the lateral resolution by minimizing the mainlobe width of the corresponding point-spread-function. However, it has not been appreciated that conventional IVUS systems with unapodized elements are particularly susceptible to image artifacts from sidelobes because of the unique geometry associated with intravascular imaging.

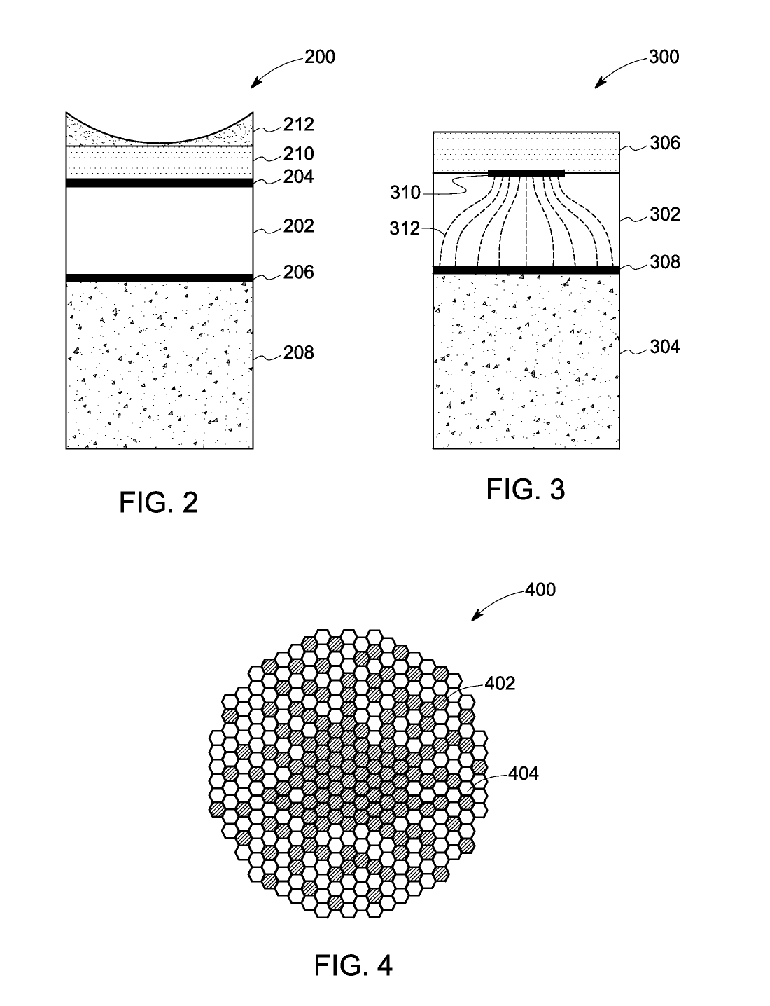

Thus, unlike conventional transducer elements that provide uniform sensitivity, the transducer 106, of the present system 100 is specifically adapted to reduce the sidelobes in the transmit and receive point-spread-functions and to improve contrast between neighboring tissue types and minimize certain image artifacts likely to occur in intravascular imaging. To that end, in one example, the transducer 106 includes one or more apodizing structures 126 that decrease the responsiveness of the transducer 106 to transmitted and received ultrasound energy at the boundaries of the transducer 106 with respect to the corresponding center. Certain exemplary embodiments of transducers including apodizing structures that allow mitigation of sidelobe artifacts in IVUS images are described in greater detail with reference to FIGS. 2-4.

FIG. 2 illustrates an exemplary embodiment of a transducer 200, such as the transducer 106 of FIG. 1, for use in improved IVUS imaging. In one embodiment, the transducer 200 corresponds to a single-element transducer for use in a catheter. Further, the transducer 200 includes a piezoelectric layer 202, for example, including a single piezoelectric crystal or a piezoelectric ceramic crystal with an associated outer electrode 204 and an associated inner electrode 206. The piezoelectric crystal, for example, includes materials such as quartz, Barium Titanate (BaTiO3) and piezoceramics such as lead zirconate titanate (PZT).

On receiving electrical excitation from a transmit circuitry, such as the transmit circuitry 114 of FIG. 1, the piezoelectric crystal vibrates at a characteristic frequency, for example the resonant frequency, which typically depends upon the thickness of the crystal. Particularly, when a voltage is applied across the piezoelectric layer 202, the thickness of the crystal expands or contracts based on the polarity of the voltage and the molecular configuration of the crystal. The expansion or contraction allows the piezoelectric layer 202 to convert electric energy into acoustic energy to generate ultrasound waves for transmission. Alternatively, the piezoelectric layer 202 converts acoustic energy corresponding to received echoes into electric signals that can be processed into useful diagnostic information.

In one embodiment, the piezoelectric layer 202 is mechanically attached to a backing layer 208 including an appropriate material such as a filled epoxy layer to absorb a part of ultrasonic energy generated by the piezoelectric layer 202. Particularly, the backing layer 208 absorbs the energy transmitted to the backside of the transducer 200 to suppress a ringing response of the transducer 200. To that end, a material having appropriate acoustic impedance and attenuation coefficient is selected for use as the backing layer 208 to avoid reverberations of the piezoelectric layer 202.

Further, the transducer 200 also includes a matching layer 210 disposed in front of the piezoelectric layer 202 to mitigate a large difference in characteristic acoustic impedance between two objects, such as the single crystal material and the soft tissues, which would otherwise result in a large reflection of the ultrasound wave at the boundary between the two objects. Particularly, in one embodiment, the transducer 200 employs, for example, a quarter-wavelength matching layer 210 that maximizes the transfer of the energy from the crystal into the tissue and from the tissue into the crystal. The matching layer 210 aims to improve the sensitivity of the transducer 200 in a desired band of frequencies, while maintaining very short pulses required for good axial imaging resolution.

Accordingly, the transducer 200 further includes one or more apodizing structures that reduce the sidelobe amplitudes in the transmit and receive point-spread-functions. In the embodiment illustrated in FIG. 2, for example, the apodizing structure is an attenuating layer 212 disposed over the matching layer 210 that smoothly increases in thickness from the center of the transducer element to the corresponding boundaries. The varying thickness of the attenuating layer 212 causes the amplitude of the emitted sound wave to drop smoothly to a small value at the element boundary when the transducer 200 is operated in transmit mode. Similarly, in receive mode, the varying thickness of the attenuating layer 212 causes the sensitivity of the transducer 200 to incident sound to decrease smoothly at the element boundary to reduce sidelobe amplitude levels.

In an alternative embodiment, however, the attenuation layer 212 may be a layer of uniform thickness for which the attenuation increases smoothly from the center of the transducer element to the corresponding boundary, for example, by varying the density of a filler material in an epoxy or polymer layer. In certain embodiments, the thickness and attenuation properties of the attenuation layer 212 are chosen to provide an attenuation of the transmitted and received ultrasound amplitude at the transducer boundary in the range of 6-60 dB, with a spatial apodization profile chosen using methods well known to those skilled in the art. Although FIG. 2 illustrates separate matching and attenuating layers, it may be noted that in certain embodiments, the matching and attenuating functions may be combined in a single layer.

Furthermore, in an embodiment where the transducer 200 corresponds to a single element transducer, the thickness of the attenuation layer 212 increases from the center of the transducer element in both dimensions of the surface of the transducer 200. However, when the transducer 200 is used as part of a one-dimensional linear transducer array, the thickness of the attenuation layer 212 increases from the center of the element in the dimension in which it is not possible to implement an apodization function electronically.

Further, FIG. 3 illustrates another exemplary embodiment of a transducer 300, such as the transducer 106 of FIG. 1, for use in improved IVUS imaging. In one embodiment, the transducer 300 includes a piezoelectric layer 302, a backing layer 304, and a matching layer 306. In certain embodiments, the structure and function of the piezoelectric layer 302, the backing layer 304, and the matching layer 306 is similar to the structure and function of the piezoelectric layer 202, the backing layer 204, and the matching layer 210, respectively, as described with reference to FIG. 2.

Additionally, the transducer 300 includes an inner electrode 308 and an outer electrode 310 associated with the piezoelectric layer 302. Typically, application of a voltage across the inner electrode 308 and the outer electrode 310 induces deformations in the layer 302 due to the piezoelectric effect. Alternatively, the deformation induced by received ultrasound energy in the piezoelectric layer 302 induces a voltage difference between the inner electrode 308 and the outer electrode 310. In certain embodiments, the spatial sensitivity of the piezoelectric ceramic layer 302 to applied voltages or received ultrasound energy is controlled by varying the geometry of the inner electrode 308 and the outer electrode 310 used in the transducer 300.

Particularly, in one embodiment, the transducer 300 includes inner and outer electrodes 308, 310 of different dimensions, such as different shapes and sizes, to generate a non-uniform electric field across the piezoelectric layer 302 when a voltage is applied. In the embodiment illustrated in FIG. 3, the outer electrode 310 in the transducer 300 is smaller than the inner electrode 308 such that density of resulting electric field lines 312 in the piezoelectric layer 302 decreases toward the element boundary.

Use of electrodes of different dimensions causes the excitation of the piezoelectric layer 302 to decrease smoothly towards the element boundary when a voltage is applied. Accordingly, the amplitude of the emitted sound waves decreases smoothly from the center of a single element, or center-line of an element in a transducer array, to a small value at the element boundaries when operating the transducer 300 in transmit mode. Similarly, when operating the transducer 300 in receive mode, the sensitivity to incident sound decreases smoothly to a small value at the element boundary, thus reducing the sidelobe amplitude levels in the transmit and receive beam patterns.

FIG. 4 illustrates another exemplary embodiment of a transducer 400, such as the transducer 106 of FIG. 1, for use in improved IVUS imaging. More specifically, FIG. 4 illustrates the face of the transducer 400, which in one example, is subdivided into hexagonal subelements. In one embodiment, only a portion of the subelements are connected to the transmit and receive circuitry, such as a connected subelement 402, shown as shaded in FIG. 4. The remaining elements, such as an unconnected subelement 404, shown as unshaded in FIG. 4, are not connected to the transmit and receive circuitry.

Further, in certain embodiments, the connected subelements 402 are arranged in a spatial pattern chosen so that the density of connected subelements 402 decreases from the center of the transducer face toward the boundaries or edges. The varying density of connected subelements 402 causes the amplitude of the emitted sound wave to drop smoothly to a small value at the transducer boundary when the transducer 400 is operated in transmit mode. Similarly, in receive mode, the varying density of connected subelements 402 causes the sensitivity of the transducer 400 to incident sound to decrease smoothly at the element boundary to reduce sidelobe amplitude levels.

FIG. 4 illustrates an exemplary hexagonal arrangement of subelements 402, 404, which is convenient when the subelements are micromachined transducers, such as capacitive micromachined ultrasound transducers (cMUT) or other structures created using microfabrication techniques. However, in certain embodiments, other shapes such as rectangular or triangular arrangements of subelements can be also be used for transducers in which the subelements are created, for example, by dicing a piezoelectric ceramic layer. Additionally, it may be noted that although FIG. 4 illustrates a transducer with a circular face, the present technique can also be applied to transducers with square, rectangular, elliptical and other shapes. The basic premise is that the spatial density of the acoustically active portion of the transducer decreases from the transducer center to its boundaries so as to provide a structural apodization means.

For brevity of the description, FIGS. 2-4 illustrate three different embodiments illustrating different apodizing means for apodizing a beam pattern used in IVUS imaging. However, in certain other embodiments, the beam pattern may be apodized to mitigate sidelobe artifacts using other apodizing means, for example, by modifying the impedance of a backing layer or modifying the density of the active piezoelectric material so as to reduce the amplitude of the emitted sound waves and reduce the sensitivity to received sound at the transducer boundary as compared to the corresponding center. In addition, the physical apodization means could be applied to one dimension of a transducer element which is part of an array of transducer elements to produce apodization in the dimension which cannot be electronically apodized.

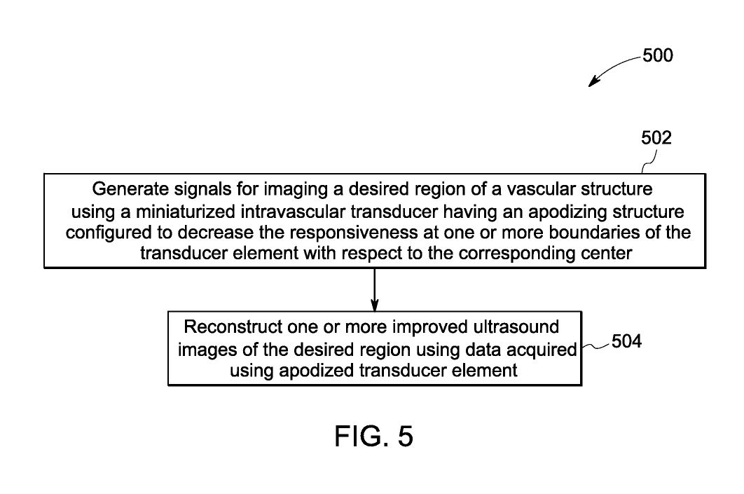

An exemplary method describing the use of apodizing means in IVUS transducers for generating substantially artifact-free intravascular images will be described in greater detail with reference of FIG. 5. Particularly, FIG. 5 illustrates a flow chart 500 depicting an exemplary method for improved intravascular ultrasound imaging using apodizing structures. The exemplary method may be described in a general context of computer executable instructions stored and/or executed on a computing system or a processor. Generally, computer executable instructions may include routines, programs, objects, components, data structures, procedures, modules, functions, and the like that perform particular functions or implement particular abstract data types. The exemplary method may also be practiced in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 5, the exemplary method is illustrated as a collection of blocks in a logical flow chart, which represents operations that may be implemented in hardware, software, or combinations thereof. The various operations are depicted in the blocks to illustrate the functions that are performed, for example, during apodization, signal transmission, and image reconstruction phases of the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIGS. 1-4.

Clinical diagnosis often relies on image-derived parameters that help a medical practitioner to identify various healthy and pathological tissue types in blood vessels. Because they use unapodized transducers, the images produced by conventional IVUS systems may contain significant image artifacts, which reduce their clinical usefulness. Low sidelobe levels are especially significant while generating diagnostic images when both strong and weak reflectors are present. In IVUS, for example, a boundary between the blood and vessel wall tends to be a strong ultrasound reflector compared with the tissue in the vessel wall. Additionally, IVUS is also used to deploy vascular stents that act as strong reflectors of ultrasound energy compared with tissue. Biodegradable stents, however, are usually difficult to visualize using ultrasound because the biodegradable stents are weak ultrasound reflectors compared with tissue. Accordingly, it is desirable to reduce artifacts caused by an interaction of the sidelobes with the strong wall reflections to generate clinically accurate images.

To that end, embodiments of the present system provide for transducer elements with one or more apodization structures that reduce sidelobe amplitudes and associated artifacts in IVUS images. Accordingly, at step 502, an intravascular transducer, such as the transducer 200, 300 or 400 of FIGS. 2-4, generate one or more ultrasound signals for imaging a desired region of a vascular structure. Particularly, the transducer includes one or more apodizing structures configured to smoothly decrease the responsiveness of the transducer element at one or more boundaries of the transducer element with respect to the center of the transducer element.

Particularly, physical apodization structures may be used to apodize the beam profile in a dimension of the transducer that cannot be electronically apodized. To that end, the apodization structures, for example, may include structures such as described with reference to FIGS. 2-4. The data acquired using the apodized transducer at step 502 can then be used to reconstruct diagnostic IVUS images of the desired portion of the vasculature, at step 504, with substantially reduced artifacts.

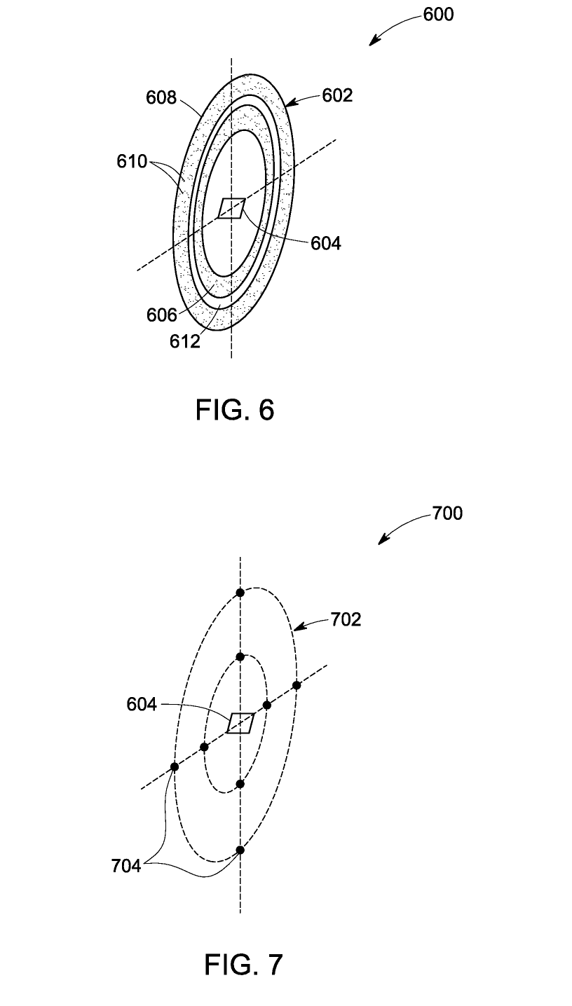

Certain exemplary simulations were performed, which revealed that apodization can produce unexpectedly large image improvements because of the approximately circular geometry of the scattering structures peculiar to IVUS imaging. FIG. 6, for example, illustrates the geometry 600 of an exemplary model 602 of a blood vessel and a rotating single-element transducer 604 used for IVUS imaging. The blood vessel is modeled as three concentric rings with the rotation axis of the transducer 604 passing through the center of the three rings, that is, through the center of the simulated blood vessel.

Particularly, the model 602 depicts the blood vessel with an inner ring 606 and an outer ring 608 that include a plurality of point-like scatterers 610 having random positions and amplitudes, along with an empty middle ring 612. The model 602, thus, approximates the ultrasound scattering properties of the intima, media, and adventia layers of an arterial wall. Further, FIG. 7 illustrates the geometry 700 of another exemplary model 702 for use in illustrating the image artifacts in the blood vessel model 602. Accordingly, the model 702 illustrates the transducer 604 depicting eight point-like scatterers 704 arranged in two circles having different radii.

The models 602 and 702 illustrated in FIGS. 6 and 7 were used to simulate corresponding ultrasound images. To that end, in one embodiment, one or more processes typical of the processing in an ultrasound imaging system used with catheter-type transducers were employed to simulate images corresponding to the models 602 and 702 to estimate improvements in image characteristics during actual IVUS imaging. The simulated images, for example, were produced by simulating acoustic propagation of a simulated ultrasound sound pulse between the transducer 604 and the simulated scatterers 610, 704, filtering a simulated received signal, and log-converting the signal envelope for each of the simulated rotational positions of the transducer 604. The set of log-converted signals for each rotational position was then scan-converted to a rectangular image format and displayed, for example, with 60 dB of dynamic range.

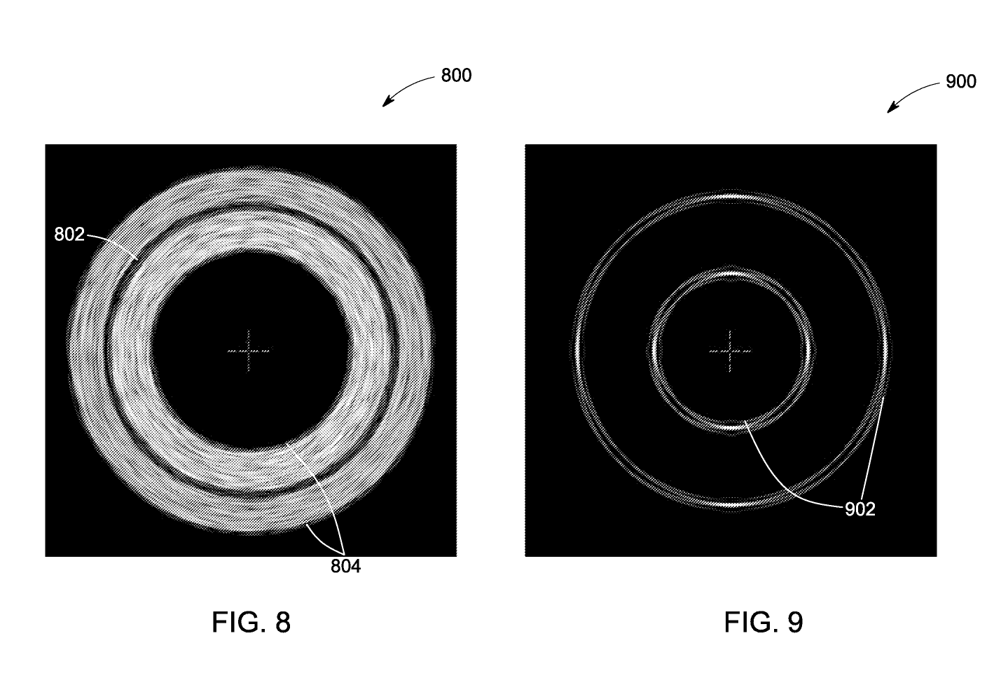

FIGS. 8 and 9 illustrate simulated images 800 and 900 produced by the models 602, 702 illustrated in FIG. 6 and FIG. 7, respectively, when the single-element transducer 604 was not apodized. In FIG. 8, some of the pixels in the simulated image 800 corresponding to the empty middle ring 612 of FIG. 6 appear bright rather than black, thus indicating presence of artifacts 802 in the simulated image 800. It was determined that the artifacts 802 were caused by the interaction of the sidelobes in the unapodized transducer's beam pattern with the scatterers 610 in the inner and outer rings 606, 608 even though the mainlobe of the beam pattern at a particular instant lay entirely in the empty middle ring 612.

Similarly, pixels 804, which correspond to depths slightly inside the inner ring 606, and slightly outside the outer ring 608, are not black because of the interaction of the sidelobes in the unapodized transducer's beam pattern with scatterers in the inner and outer rings 606, 608 even when the mainlobe at a particular instant lay entirely inside the inner ring 606 or entirely outside the outer ring 608. Further, FIG. 9 illustrates a simulated image 900 of the point-like scatterers 704 of FIG. 7 produced by the transducer 604, when unapodized. In FIG. 9, the sidelobes of the unapodized transducer's beam pattern are clearly visible as “tails” attached to the bright pixels corresponding to the scatterers 610 in FIG. 6. In FIG. 9, it may be noted that the image of the sidelobes' interaction with the scatterers 704 shows that the sidelobe artifact 902 appears at radii slightly smaller and slightly larger than the radii of the scatterers 704, which explains the presence of non-black pixels in FIG. 8 in regions where there were no scatterers in the simulated blood vessel, as depicted in FIG. 6.

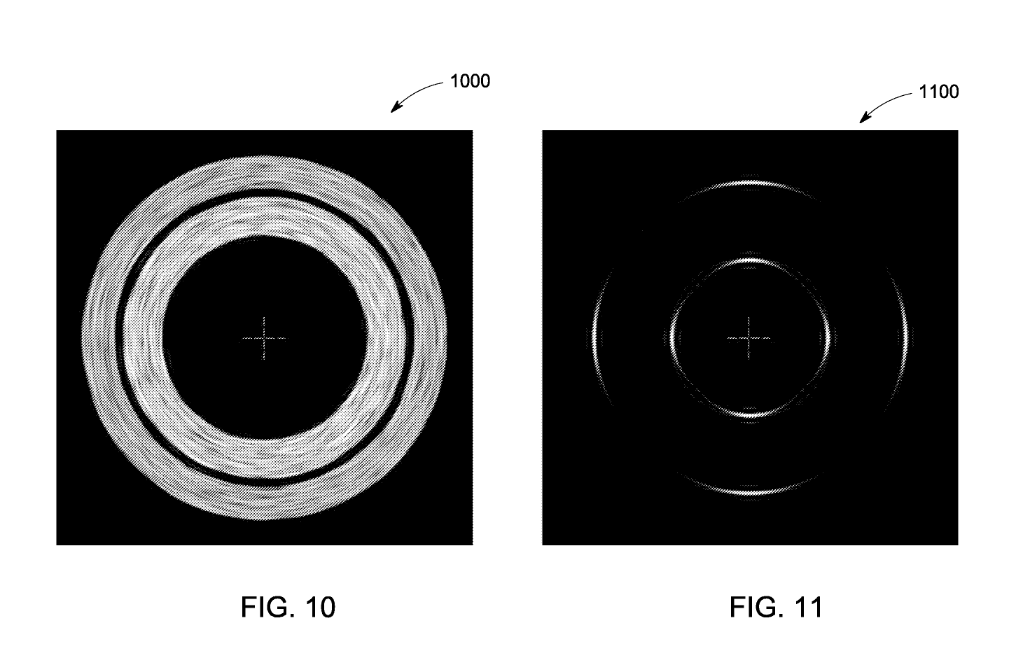

In contrast to the images 800 and 900 generated using the unapodized transducer 604, FIGS. 10 and 11 illustrate simulated images generated using the transducer 604 including apodizing means. FIG. 10 illustrates a simulated image 1000 corresponding to the model 602 of FIG. 6 and produced by the transducer 604 when using apodizing means such as the attenuation layer 312 of FIG. 3. Particularly, the simulations showed that use of the apodizing means reduces the artifacts described with reference to FIG. 8 and FIG. 9. The image 1000 of FIG. 10, thus, more closely corresponds to the structure of the simulated scatterers 610 of FIG. 6 than the image 800 of FIG. 8. Similarly, FIG. 11 illustrates the image 1100 simulated by the apodized transducer 604 using the model 702 of FIG. 7. In comparison to the image 900 of FIG. 9 generated using the unapodized transducer 604, the sidelobe artifact depicted in image 1100 is much lower, so that the simulated image 1100 more closely corresponds to the scatter properties in the mainlobe of the transducer's beam profile.

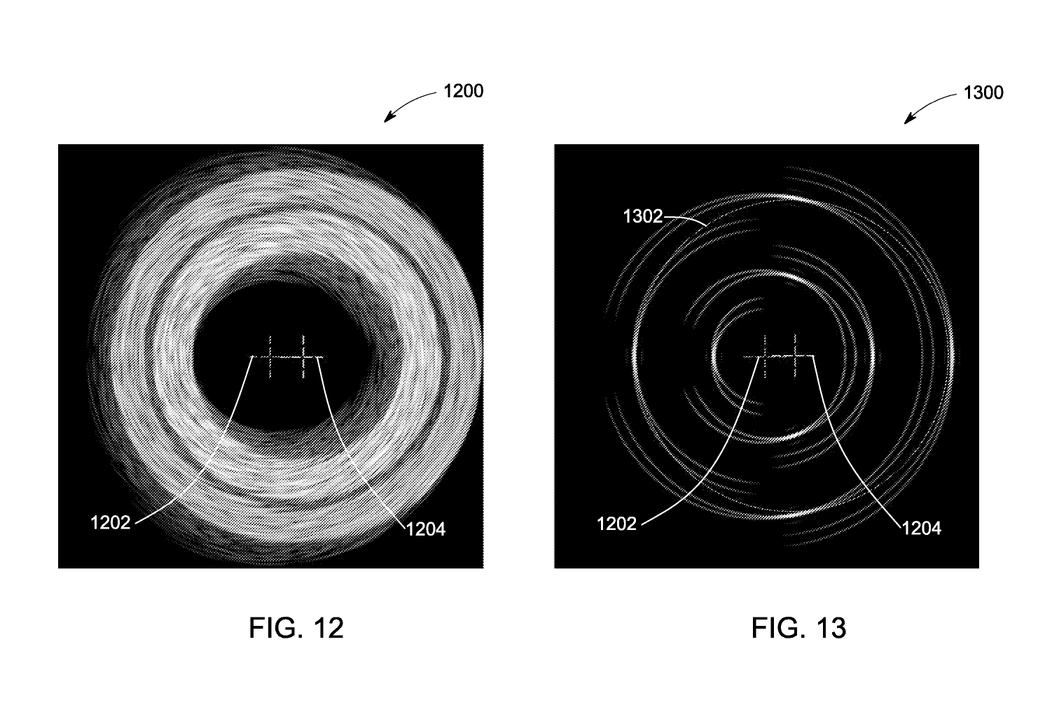

Further, FIG. 12 illustrates a simulated image 1200 of the scatterers 610 in FIG. 6 generated with the unapodized transducer 604 when the transducer rotation axis 1202 was not aligned with the center 1204 of the simulated blood vessel. Particularly, FIG. 12 depicts that the sidelobe artifact is much more pronounced when the transducer rotation axis 1202 is not centered in the blood vessel than when it is centered, as depicted in FIG. 8. Similarly, FIG. 13 illustrates a simulated image 1300 of the scatterers 704 in FIG. 7 generated with the unapodized transducer 704 when the transducer axis 1202 is offset from the center 1204 of the point-like scatterers 704. A circle 1302 was overlaid on the image 1300 in FIG. 13. The circle 1302 had a radius slightly less than the radius of the circle on which the outer four scatterers 704 of FIG. 7 lay. Particularly, from FIG. 13, it is evident that in absence of apodization, the radial offset of the sidelobes compared with the target geometry (the model 702), is much more severe when the transducer rotation axis 1202 does not coincide with the center of the target geometry 1204, thus resulting in larger sidelobe artifacts.

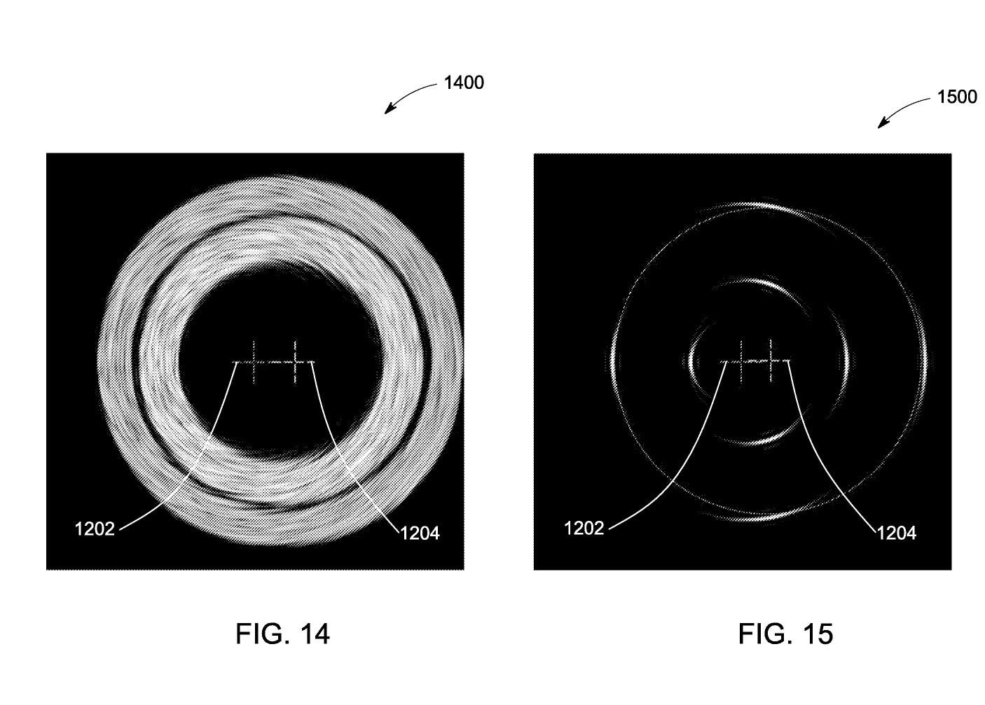

In contrast to images 1200 and 1300, FIGS. 14 and 15 illustrate simulated images 1400 and 1500 generated using the apodized transducer 604 when the rotation axis 1202 of the transducer 604 was not aligned with the center 1204 of simulated blood vessel. As depicted by FIGS. 14 and 15, the reduction is sidelobe amplitude levels generated by apodizing the transducer 604 reduces the image artifacts considerably even when the transducer axis 1202 is offset from the center 1204 of the simulated blood vessel.

Embodiments of the present systems and methods, thus, may be employed to generate ultrasound images with significantly reduced artifacts by apodizing miniaturized IVUS transducer elements. Particularly, the embodiments described herein aid in generating high quality ultrasound images that allow accurate assessment of clinically relevant parameters for use in diagnosis and treatment of various health conditions of a patient.

While only certain features of the present invention have been illustrated and described herein, many modifications and changes will occur to those skilled in the art. It is, therefore, to be understood that the appended claims are intended to cover all such modifications and changes as fall within the true spirit of the invention.

1. An intravascular ultrasound catheter, comprising:
at least one miniaturized transducer element adapted for use in an intravascular region of interest of a subject, wherein the miniaturized transducer element is configured to produce signals for use in generating one or more ultrasound images of the intravascular region of interest of the subject, and
one or more apodizing structures operably connected to the transducer element, wherein the apodizing structures are configured to decrease the responsiveness of the transducer element at one or more boundaries of the transducer element with respect to the center of the transducer element for reducing sidelobe amplitude of an ultrasound beam profile in the region of interest, thereby generating improved ultrasound images.

2. The transducer of claim 1, wherein the dimension of the transducer element is 0.5 millimeters.

3. The transducer of claim 1, wherein the dimension of the transducer element is in the range of 0.25 to 1.0 millimeters.

4. An intravascular ultrasound imaging system comprising:
a catheter comprising at least one miniaturized transducer element adapted to be inserted into a vascular structure and configured to produce signals for use in generating one or more ultrasound images of a desired region within the vascular structure;
one or more apodizing structures operably coupled to the transducer element, wherein the apodizing structures are configured to decrease the responsiveness of the transducer element at one or more boundaries of the transducer element with respect to the center of the transducer element for reducing a sidelobe amplitude of an ultrasound beam profile used for imaging the desired region; and
a processing unit operably coupled to the catheter, wherein the processing unit processes the signals generated by the transducer element for generating the one or more ultrasound images of the desired region within the vascular structure such that the one or more ultrasound images have reduced artifacts.

5. The system of claim 4, wherein the one or more apodizing structures comprise an attenuation layer mechanically attached to a surface of the transducer element, and wherein thickness of the attenuation layer increases from the center of the transducer element towards the one or more boundaries of the transducer element.

6. The system of claim 5, wherein the attenuation layer comprises a filled epoxy layer, a polymer layer, or a combination thereof.

7. The system of claim 5, wherein the thickness of the attenuation layer varies so as to produce attenuation of 6-60 decibels at the one or more boundaries of the transducer element with respect to the center of the transducer element.

8. The system of claim 4, wherein the one or more apodizing structures comprise an attenuation layer of uniform thickness mechanically attached to a surface of the transducer element, and wherein absorption of the attenuation layer increases from the center of the transducer element towards the one or more boundaries of the transducer element.

9. The system of claim 4, wherein the one or more apodizing structures comprise a matching layer in the transducer element.

10. The system of claim 4, wherein the one or more apodizing structures comprise a plurality of electrodes having different dimensions and associated with the transducer element.

11. The system of claim 10, wherein the dimensions of the plurality of electrodes are adapted to generate a plurality of electric field lines such that density of the electric field lines in the transducer element decreases from the center of the transducer element towards the one or more boundaries of the transducer element.

12. The system of claim 4, wherein the one or more apodizing structures are configured to apodize a dimension of the transducer element that cannot be electronically apodized.

13. An ultrasound imaging method, comprising:
generating one or more signals for imaging a desired region of a vascular structure using an intravascular transducer, wherein the transducer comprises at least one miniaturized transducer element and one or more apodizing structures configured to decrease the responsiveness of the transducer element at one or more boundaries of the transducer element with respect to the center of the transducer element for reducing a sidelobe amplitude of an ultrasound beam profile used for imaging the desired region; and
reconstructing one or more improved ultrasound images of the desired region using data acquired using the apodized transducer element.

14. The method of claim 13, wherein the one or more apodization structures comprise an absorption layer.

15. The method of claim 14, wherein thickness of the absorption layer increases from the center of the transducer element towards one or more boundaries of the transducer element.

16. The method of claim 14, further comprising using the absorption layer to apodize a dimension of the transducer that cannot be electronically apodized.

17. The method of claim 13, wherein the one or more apodization structures comprise a pair of electrodes.

18. The method of claim 17, further comprising varying one or more structural specifications of the pair of electrodes for decreasing an internal electric field in the transducer element from the corresponding center towards one or more boundaries of the transducer element.

19. The method of claim 18, wherein decreasing the internal electric field comprises decreasing emitted sound pressure on transmit from one or more boundaries of the transducer element with respect to the corresponding center.

20. The method of claim 18, wherein decreasing the internal electric field comprises decreasing sensitivity to sound pressure on receive from one or more boundaries of the transducer element with respect to the corresponding center.

21. The method of claim 13, wherein the at least one miniaturized transducer element is subdivided into one or more subelements, and wherein the one or more apodization structures comprise one or more of the subelements arranged in a specific spatial pattern such that the apodization structures are configured to decrease the responsiveness of the transducer element at one or more boundaries of the transducer element with respect to the center of the transducer element.

22. The method of claim 13, further comprising modifying density of an active piezoelectric material of the transducer element for apodizing the transducer.

23. The method of claim 13, wherein the one or more apodization structures comprise a backing layer in the transducer element.

24. The method of claim 23, further comprising modifying the impedance of the backing layer for apodizing the transducer.
